(19) United States
(12) Patent Application Publication
(10) Pub. No.: US 2026/0260739 A1
(43) Pub. Date: Sep. 3, 2026

(54) SYSTEMS AND METHODS FOR OPTIMIZING LEARNER VOLUME AT AN INSTITUTION

(71) Applicant: MedEd Cartography, LLC, Hudson Oaks, TX (US)

(72) Inventors: Dustin DeMoss, Aledo, TX (US); Brittni Brown, Weatherford, TX (US)

(73) Assignee: MedEd Cartography, LLC, Hudson Oaks, TX (US)

(21) Appl. No.: 19/552,333

(22) Filed: Feb. 27, 2026

Related U.S. Application Data
(60) Provisional application No. 63/765,056, filed on Feb. 28, 2025.

(51) Int. Cl. G16H 40/20

(52) U.S. Cl. CPC G16H 40/20

(57) ABSTRACT

A method for optimizing learner volume at an institution includes accessing data indicating a maximum capacity of simultaneous learners for each team of a plurality of teams. The method further includes accessing anticipated learner data that includes a plurality of learners to be schedule, and a unique learner value assigned to each particular learner of the plurality of learners to be scheduled. The method further includes automatically calculating a projected learner value for each particular team of the plurality of teams using the anticipated learner data. The method further includes automatically calculating an available learner capacity for each particular team of the plurality of teams using the calculated projected learner value for each particular team. The method further includes displaying, on an electronic display, the calculated available learner capacity for at least one team of the plurality of teams.

### PRIORITY

This application claims the benefit, under 35 U.S.C. § 119(e), of U.S. Provisional Patent Application No. 63/765,056, filed Feb. 28, 2025, the entirety of which is herein incorporated by reference for all purposes.

### TECHNICAL FIELD

This disclosure generally relates to scheduling learners, and more specifically to systems and methods for optimizing learner volume at an institution.

### BACKGROUND

Scheduling personnel is a challenging process for many institutions. In the medical industry, for example, personnel known as learners (e.g., students, residents, fellows, etc.) are typically scheduled for clinical experiences with various preceptors (e.g., attending physicians, experienced advanced practice providers, supervising psychologists, etc.) within a medical facility or campus. Each preceptor typically has a certain learner capacity that may vary at different times of the year based on multiple different factors. For example, a preceptor may have a certain capacity for learners in one month but a different capacity in another month based on the types of learners that the preceptor is overseeing for those time periods. These and other factors cause the scheduling of learners for clinical experiences with preceptors to be challenging and time consuming.

### SUMMARY

The present disclosure achieves technical advantages as systems, methods, and computer-readable storage media for optimizing learner volume at an institution. The present disclosure provides for a system integrated into a practical application with meaningful limitations that may include automatically optimizing learner volume at an institution and displaying a 3D learner capacity map on an electronic display. Other meaningful limitations of the system integrated into a practical application include automatically providing one or more alerts/notifications on a computing system as described herein.

The present disclosure solves the technological problem of a lack of technical functionality for optimizing learner volume at an institution. The technological solutions provided herein, and missing from conventional systems, are more than a mere application of a manual process to a computerized environment, but rather include functionality (including artificial intelligence functionality) to implement a technical process to supplement current manual solutions for optimizing learner volume at an institution. In doing so, the present disclosure goes well beyond a mere application the manual process to a computer.

Unlike existing solutions where personnel may be required to manually determine learner volume at an institution, embodiments of this disclosure provide systems and methods that provide functionality for automatically and optimally determining learner volume at an institution. By automatically and optimally determining learner volume at an institution, the efficiency of the learner scheduling environment of a medical facility may be increased and bandwidth of personnel typically tasked with scheduling learners may be increased. Other technical advantages will be readily apparent to one skilled in the art from the following figures, descriptions, and claims. Moreover, while specific advantages have been enumerated above, various embodiments may include all, some, or none of the enumerated advantages.

In some embodiments, the disclosed models are formulated or otherwise configured to utilize various constraints and objectives in order to perform or execute a designated task. In other embodiments, the present disclosure includes techniques for implementing and training models (e.g., machine-learning models, artificial intelligence models, algorithmic constructs, optimizers, etc.) for performing or executing a designated task or a series of tasks. In these embodiments, the disclosed techniques provide a systematic approach for the training of such models to enhance performance, accuracy, and efficiency in their respective applications. In embodiments, the techniques for training the models can include collecting a set of data from a database, conditioning the set of data to generate a set of conditioned data, and/or generating a set of training data including the collected set of data and/or the conditioned set of data.

In some embodiments, a model can undergo a training phase wherein the model may be exposed to the set of training data, such as through an iterative processes of learning in which the model adjusts and optimizes its parameters and algorithms to improve its performance on the designated task or series of tasks. This training phase may configure the model to develop the capability to perform its intended function with a high degree of accuracy and efficiency. In embodiments, the conditioning of the set of data may include modification, transformation, and/or the application of targeted algorithms to prepare the data for training. The conditioning step may be configured to ensure that the set of data is in an optimal state for training the model, resulting in an enhancement of the effectiveness of the model's learning process. These features and techniques not only qualify as patent-eligible features but also introduce substantial improvements to the field of computational modeling. These features are not merely theoretical but represent an integration of a concepts into practical applications that significantly enhance the functionality, reliability, and efficiency of the models developed through these processes.

In embodiments, the present disclosure includes techniques for generating a notification of an event (e.g., an output notification, a user notification such as a text message, an email, etc.) that includes generating an alert that includes information specifying the location of a source of data associated with the event, formatting the alert into data structured according to an information format, and transmitting the formatted alert over a network to a device associated with a receiver based upon a destination address and a transmission schedule. In embodiments, receiving the alert enables a connection from the device associated with the receiver to the data source over the network when the device is connected to the source to retrieve the data associated with the event and causes a viewer application (e.g., a graphical user interface (GUI)) to be activated (e.g., via a hyperlink) to display the data associated with the event. These features represent patent eligible features, as these features amount to significantly more than an abstract idea.

Such features, when considered as an ordered combination, amount to significantly more than simply organizing and comparing data. The features address the Internet-centric challenge of alerting a receiver with time sensitive information. This is addressed by transmitting the alert over a network to activate the viewer application, which enables the connection of the device of the receiver to the source over the network to retrieve the data associated with the event. These are meaningful limitations that add more than generally linking the use of an abstract idea (e.g., the general concept of organizing and comparing data) to the Internet, because they solve an Internet-centric problem with a solution that is necessarily rooted in computer technology. These features, when taken as an ordered combination, provide unconventional steps that confine the abstract idea to a particular useful application. Therefore, these features represent patent eligible subject matter.

Moreover, in embodiments, one or more operations and/or functionality of components described herein can be distributed across a plurality of computing systems (e.g., personal computers (PCs), user devices, servers, processors, etc.), such as by implementing the operations over a plurality of computing systems. This distribution can be configured to facilitate the optimal load balancing of requests, which can encompass a wide spectrum of network traffic or data transactions. By leveraging a distributed operational framework, a system implemented in accordance with embodiments of the present disclosure can effectively manage and mitigate potential bottlenecks, ensuring equitable processing distribution and preventing any single device from shouldering an excessive burden. This load balancing approach significantly enhances the overall responsiveness and efficiency of the network, markedly reducing the risk of system overload and ensuring continuous operational uptime. The technical advantages of this distributed load balancing can extend beyond mere efficiency improvements. It introduces a higher degree of fault tolerance within the network, where the failure of a single component does not precipitate a systemic collapse, markedly enhancing system reliability.

Additionally, this distributed configuration promotes a dynamic scalability feature, enabling the system to adapt to varying levels of demand without necessitating substantial infrastructural modifications. The integration of advanced algorithmic strategies for traffic distribution and resource allocation can further refine the load balancing process, ensuring that computational resources are utilized with optimal efficiency and that data flow is maintained at an optimal pace, regardless of the volume or complexity of the requests being processed. Moreover, the practical application of these disclosed features represents a significant technical improvement over traditional centralized systems. Through the integration of the disclosed technology into existing networks, entities can achieve a superior level of service quality, with minimized latency, increased throughput, and enhanced data integrity. The distributed approach of embodiments not only bolster the operational capacity of computing networks but offer a robust framework for the development of future technologies, underscoring its value as a foundational advancement in the field of network computing.

Further, to aid in the load balancing, the computing system can spawn multiple processes and threads to process data concurrently. The speed and efficiency of the computing system can be greatly improved by instantiating more than one process or thread to implement the claimed functionality. However, one skilled in the art of programming will appreciate that use of a single process or thread can also be utilized and is within the scope of the present disclosure.

Accordingly, the present disclosure discloses concepts inextricably tied to computer technology such that the present disclosure provides the technological benefit of implementing functionality to automatically and optimally determine learner volume an institution. The systems and techniques of embodiments provide improved systems by providing capabilities to perform functions that are currently performed manually and to perform functions that are currently not possible.

In some embodiments, a system includes one or more memory units and one or more computer processors communicatively coupled to the one or more memory units. The one or more memory units are configured to store data indicating a maximum capacity of simultaneous learners for each team of a plurality of teams. The one or more memory units are further configured to store anticipated learner data that includes a plurality of learners to be scheduled and a unique learner value assigned to each particular learner of the plurality of learners to be scheduled. The one or more computer processors are configured to access the data indicating the maximum capacity of simultaneous learners for each team of the plurality of teams. The one or more computer processors are further configured to access the anticipated learner data. The one or more computer processors are further configured to automatically calculate a projected learner value for each particular team of the plurality of teams using the anticipated learner data. The one or more computer processors are further configured to automatically calculate an available learner capacity for each particular team of the plurality of teams using the calculated projected learner value for each particular team. The one or more computer processors are further configured to display, on an electronic display, the calculated available learner capacity for at least one team of the plurality of teams.

In some embodiments, a method for optimizing learner volume at an institution includes accessing data indicating a maximum capacity of simultaneous learners for each team of a plurality of teams. The method further includes accessing anticipated learner data that includes a plurality of learners to be schedule, and a unique learner value assigned to each particular learner of the plurality of learners to be scheduled. The method further includes automatically calculating a projected learner value for each particular team of the plurality of teams using the anticipated learner data. The method further includes automatically calculating an available learner capacity for each particular team of the plurality of teams using the calculated projected learner value for each particular team. The method further includes displaying, on an electronic display, the calculated available learner capacity for at least one team of the plurality of teams.

In some embodiments, one or more computer-readable non-transitory storage media embodies instructions that, when executed by a processor, cause the processor to perform operations including accessing data indicating a maximum capacity of simultaneous learners for each team of a plurality of teams. The operations further include accessing anticipated learner data comprising a plurality of learners to be scheduled and a unique learner value assigned to each particular learner of the plurality of learners to be scheduled. The operations further include automatically calculating a projected learner value for each particular team of the plurality of teams using the anticipated learner data. The operations further include automatically calculating an available learner capacity for each particular team of the plurality of teams using the calculated projected learner value for each particular team. The operations further include displaying, on an electronic display, the calculated available learner capacity for at least one team of the plurality of teams.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present disclosure and its advantages, reference is now made to the following descriptions taken in conjunction with the accompanying drawings, in which:

FIG. 1 is a diagram illustrating a learner volume optimization system, according to particular embodiments.

FIG. 2 is a diagram illustrating a maximum capacity of simultaneous learners for a particular division of an institution, according to particular embodiments.

FIGS. 3A and 3B are diagrams illustrating anticipated learner data for a particular division of an institution, according to particular embodiments.

FIGS. 4A and 4B are diagrams illustrating a projected learner capacity for a particular division of an institution, according to particular embodiments.

FIGS. 5A and 5B are diagrams illustrating a projected learner capacity dashboard that may be generated and displayed by the learner volume optimization system of FIG. 1, according to particular embodiments.

FIG. 6A is a diagram illustrating a three-dimensional (3D) learner capacity map that may be generated and displayed by the learner volume optimization system of FIG. 1, according to particular embodiments.

FIG. 6B is a chart illustrating a method for optimizing learner volume at an institution by the learner volume optimization system of FIG. 1, according to particular embodiments.

FIG. 7 is an example computer system that can be utilized to implement aspects of the various technologies presented herein, according to particular embodiments.

It should be understood that the drawings are not necessarily to scale and that the disclosed embodiments are sometimes illustrated diagrammatically and in partial views. In certain instances, details which are not necessary for an understanding of the disclosed methods and apparatuses or which render other details difficult to perceive may have been omitted. It should be understood, of course, that this disclosure is not limited to the particular embodiments illustrated herein.

### DETAILED DESCRIPTION

The disclosure presented in the following written description and the various features and advantageous details thereof, are explained more fully with reference to the non-limiting examples included in the accompanying drawings and as detailed in the description. Descriptions of well-known components have been omitted to not unnecessarily obscure the principal features described herein. The examples used in the following description are intended to facilitate an understanding of the ways in which the disclosure can be implemented and practiced. A person of ordinary skill in the art would read this disclosure to mean that any suitable combination of the functionality or exemplary embodiments below could be combined to achieve the subject matter claimed. The disclosure includes either a representative number of species falling within the scope of the genus or structural features common to the members of the genus so that one of ordinary skill in the art can recognize the members of the genus. Accordingly, these examples should not be construed as limiting the scope of the claims.

A person of ordinary skill in the art would understand that any system claims presented herein encompass all of the elements and limitations disclosed therein, and as such, require that each system claim be viewed as a whole. Any reasonably foreseeable items functionally related to the claims are also relevant. The Examiner, after having obtained a thorough understanding of the disclosure and claims of the present application has searched the prior art as disclosed in patents and other published documents, i.e., nonpatent literature. Therefore, as evidenced by issuance of this patent, the prior art fails to disclose or teach the elements and limitations presented in the claims as enabled by the specification and drawings, such that the presented claims are patentable under the applicable laws and rules of this jurisdiction.

Scheduling personnel is a challenging process for many institutions. In the medical industry, for example, personnel known as learners (e.g., students, residents, fellows, etc.) are typically scheduled for clinical experiences with various preceptors within a medical facility or campus. Each preceptor typically has a certain learner capacity that may vary at different times of the year based on multiple different factors. For example, a preceptor may have a certain capacity for learners in one month but a different capacity in another month based on the types of learners that the preceptor is overseeing for those time periods. These and other factors cause the scheduling of learners for clinical experiences with preceptors to be challenging and time consuming.

To address these and other problems with scheduling personnel such as learners at an institution such as a medical campus, the disclosed embodiments provide systems and methods for automatically and optimally determining and displaying learner volume at an institution. For example, certain embodiments of the disclosure access various input data regarding available learner capacity (e.g., maximum capacity of simultaneous learners, anticipated learner data, etc.), analyze the input data, and then generate one or more outputs to display projected learner capacity for the institution. The outputs generally provide an indication of learner capacity for particular groups/divisions/preceptors for a particular time period. For example, certain embodiments display a 3D learner capacity map that visually indicates available capacity for learners for an institution using a 3D map. As a result, administrators and managers of learners as well as preceptors may have an accurate and readily-accessible indication of learner capacity.

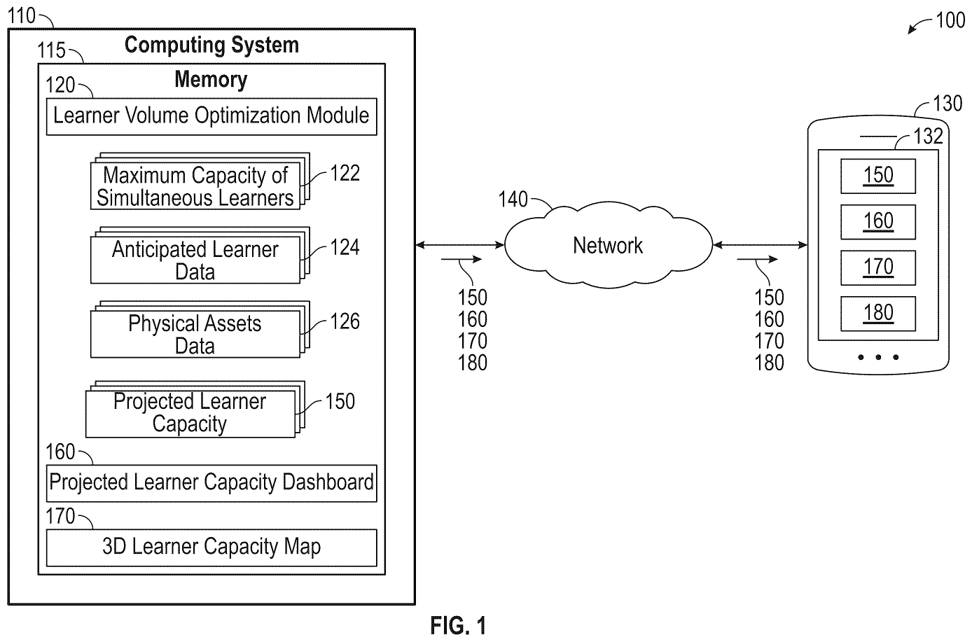

FIG. 1 is a diagram illustrating a learner volume optimization system 100, according to particular embodiments. Learner volume optimization system 100 includes a computing system 110, a learner volume optimization module 120, a client system 130, and a network 140. Computing system 110, learner volume optimization module 120, and client system 130 are all communicatively coupled with each other using any appropriate wired or wireless communication system or network (e.g., network 140). Computing system 110 includes a computer processor (e.g., processor 702) and memory 115 (e.g., memory 704) that stores learner volume optimization module 120. Client system 130 includes an electronic display for displaying a user interface 132.

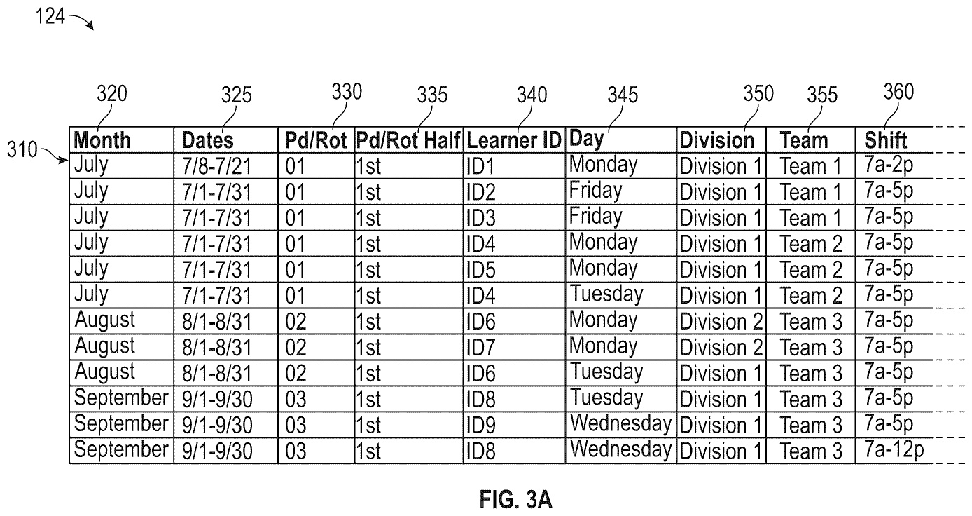

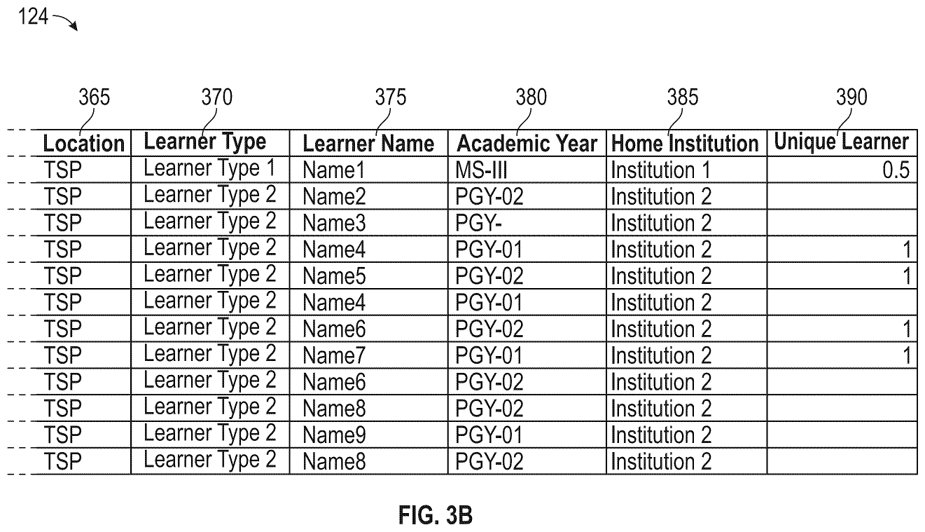

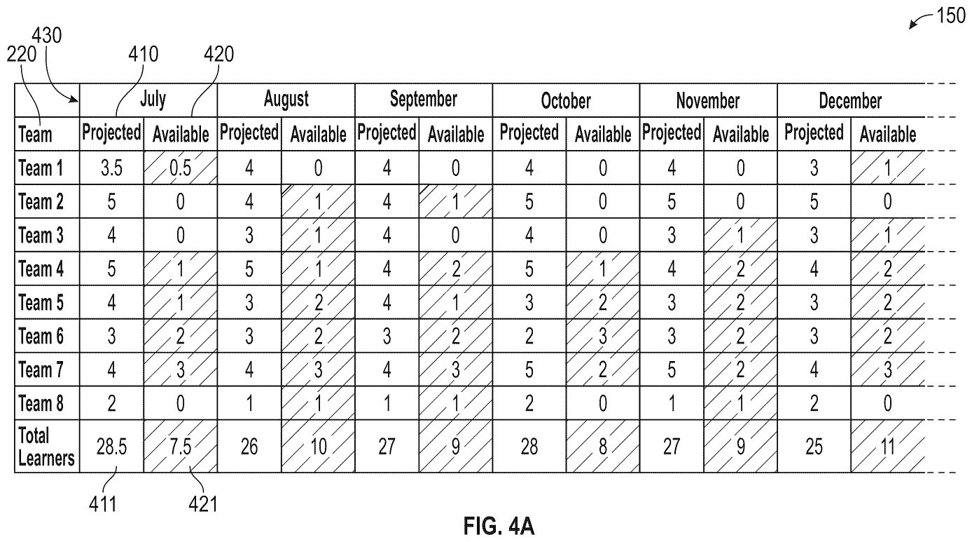

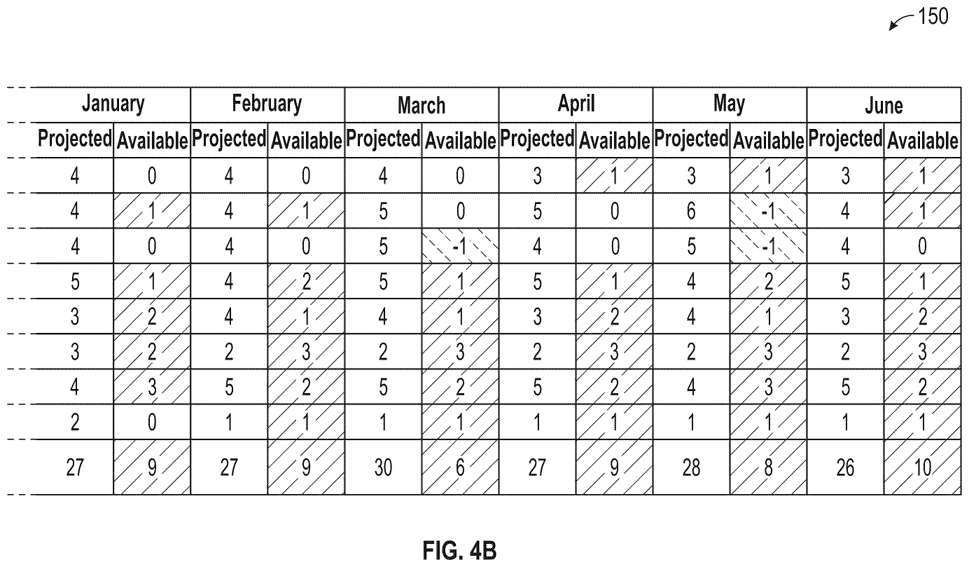

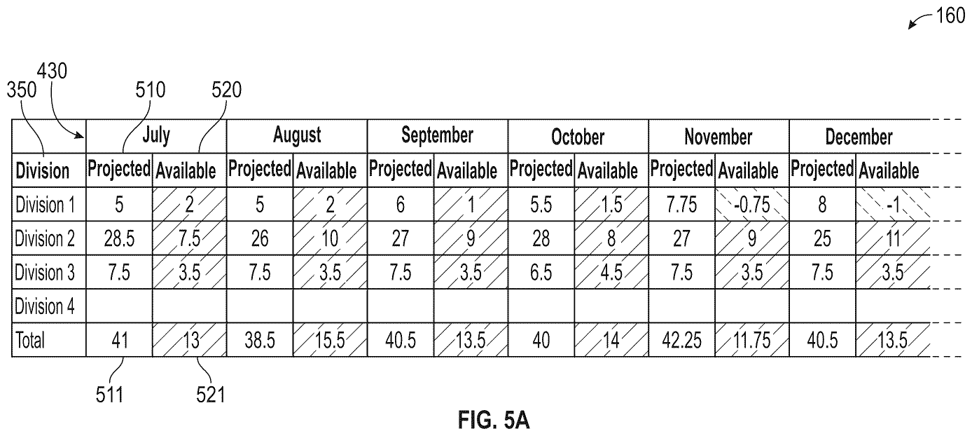

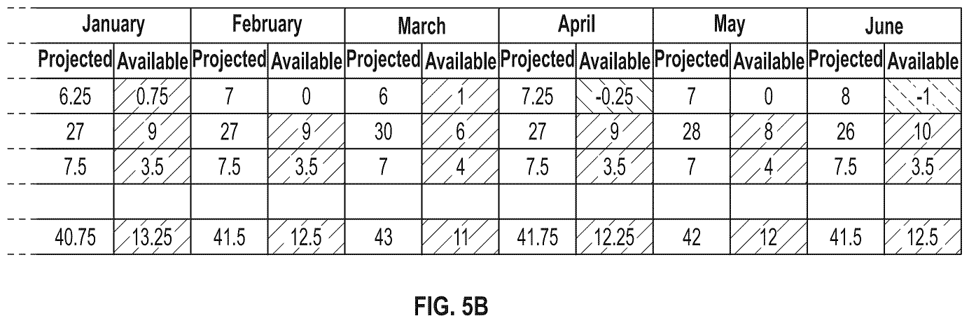

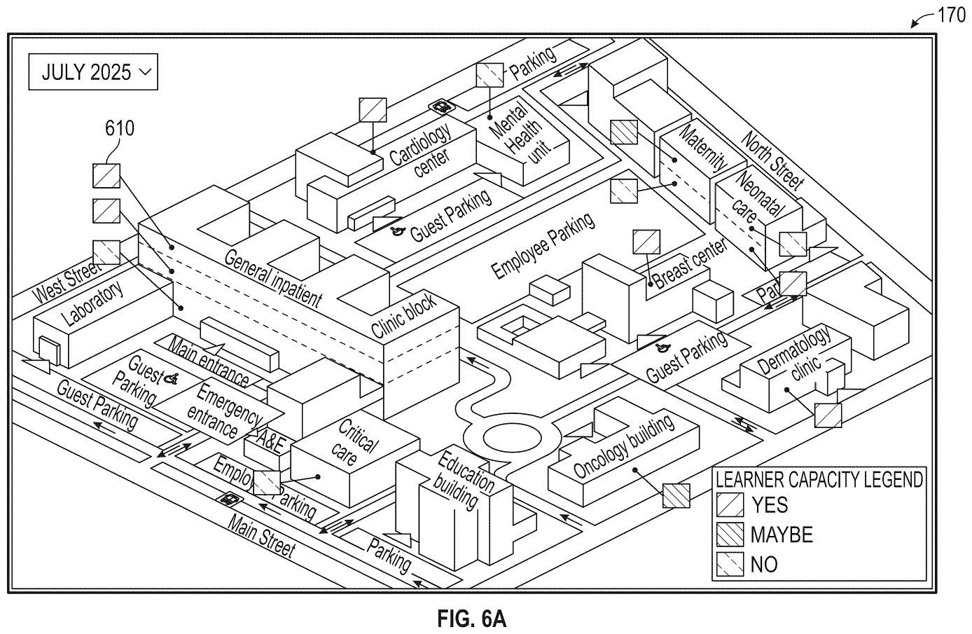

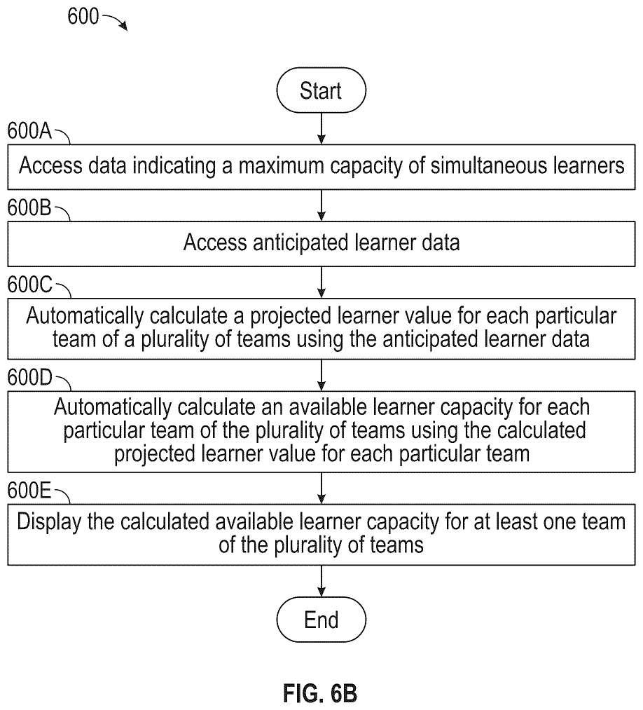

In general, learner volume optimization system 100 receives or otherwise accesses various input data (e.g., maximum capacity of simultaneous learners 122, anticipated learner data 124, physical assets data 126, etc.) and analyzes the input data in order generate one or more outputs (e.g., projected learner capacity 150 as illustrated in FIGS. 4A and 4B, projected learner capacity dashboard 160 as illustrated in FIGS. 5A and 5B, and 3D learner capacity map 170 as illustrated in FIG. 6A). The outputs provided by learner volume optimization system 100 generally provide an indication of learner capacity for particular groups/divisions/preceptors for a particular time period. For example, certain embodiments of projected learner capacity 150 visually indicate available capacity for learners for one or more teams of a particular division of the institution. As another example, certain embodiments of projected learner capacity dashboard 160 visually indicate available capacity for learners for multiple divisions of the institution. As yet another example, certain embodiments of 3D learner capacity map 170 visually indicate available capacity for learners for an institution using a 3D map. As a result, administrators and managers of learners as well as preceptors may have an accurate and readily-accessible view of current and future learner capacity.

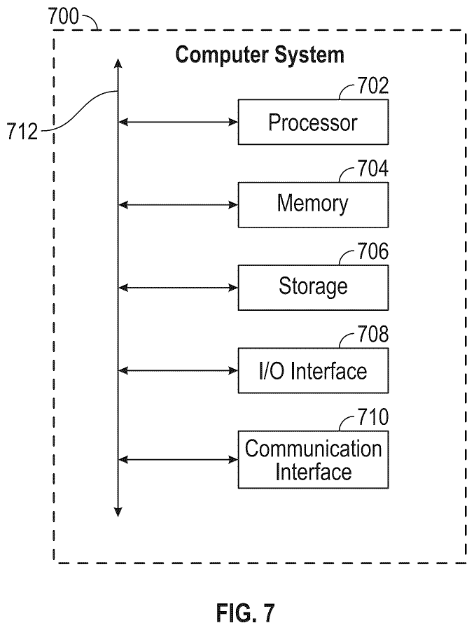

Computing system 110 may be any appropriate computing system in any suitable physical form. As example and not by way of limitation, computing system 110 may be an embedded computer system, a system-on-chip (SOC), a single-board computer system (SBC) (such as, for example, a computer-on-module (COM) or system-on-module (SOM)), a desktop computer system, a laptop or notebook computer system, a mainframe, a mesh of computer systems, a mobile telephone, a personal digital assistant (PDA), a server, a tablet computer system, an augmented/virtual reality device, or a combination of two or more of these. Where appropriate, computing system 110 may include one or more computer systems; be unitary or distributed; span multiple locations; span multiple machines; span multiple data centers; or reside in a cloud, which may include one or more cloud components in one or more networks. Where appropriate, computing system 110 may perform without substantial spatial or temporal limitation one or more steps of one or more methods described or illustrated herein. As an example, and not by way of limitation, computing system 110 may perform in real time or in batch mode one or more steps of one or more methods described or illustrated herein. Computing system 110 may perform at different times or at different locations one or more steps of one or more methods described or illustrated herein, where appropriate. A particular example of a computing system 110 is described in reference to FIG. 7.

Computing system 110 includes one or more memory units/devices 115 (collectively herein, “memory 115”) that may store learner volume optimization module 120. Learner volume optimization module 120 may be a software module/application utilized by computing system 110 to generate and provide projected learner capacity 150, projected learner capacity dashboard 160, and 3D learner capacity map 170, as described herein. Learner volume optimization module 120 represents any suitable set of instructions, logic, or code embodied in a computer-readable storage medium. For example, learner volume optimization module 120 may be embodied in memory 115, a disk, a CD, or a flash drive. In particular embodiments, learner volume optimization module 120 may include instructions (e.g., a software application) executable by a computer processor to perform some or all of the functions described herein.

Client system 130 is any appropriate user device for communicating with components of learner volume optimization system 100 over network 140 (e.g., the internet). In particular embodiments, client system 130 may be an electronic device including hardware, software, or embedded logic components or a combination of two or more such components and capable of carrying out the appropriate functionalities implemented or supported by client system 130. As an example, and not by way of limitation, a client system 130 may include a computer system (e.g., computer system 700) such as a desktop computer, notebook or laptop computer, netbook, a tablet computer, e-book reader, GPS device, camera, personal digital assistant (PDA), handheld electronic device, cellular telephone, smartphone, smartwatch, augmented/virtual reality device such as wearable computer glasses, other suitable electronic device, or any suitable combination thereof. This disclosure contemplates any suitable client system 130. A client system 130 may enable a network user at client system 130 to access network 140. A client system 130 may enable a user to communicate with other users at other client systems 130. Client system 130 may include an electronic display that displays graphical user interface 132, a processor such processor 702, and memory such as memory 704.

Network 140 allows communication between and amongst the various components of learner volume optimization system 100. This disclosure contemplates network 140 being any suitable network operable to facilitate communication between the components of learner volume optimization system 100. Network 140 may include any interconnecting system capable of transmitting audio, video, signals, data, messages, or any combination of the preceding. Network 140 may include all or a portion of a local area network (LAN), a wide area network (WAN), an overlay network, a software-defined network (SDN), a virtual private network (VPN), a packet data network (e.g., the Internet), a mobile telephone network (e.g., cellular networks, such as 4G or 5G), a Plain Old Telephone (POT) network, a wireless data network (e.g., WiFi, WiGig, WiMax, etc.), a Long Term Evolution (LTE) network, a Universal Mobile Telecommunications System (UMTS) network, a peer-to-peer (P2P) network, a Bluetooth network, a Near Field Communication network, a Zigbee network, and/or any other suitable network.

As used herein, “learner” may refer to any personnel that is to be scheduled with and/or assigned to any group or entity for any predetermined amount of time. In some embodiments, “learner” refers to medical personnel such as students (e.g., medical students, physician assistant (PA) students, nurse practitioner students, psychology students, etc.), residents, fellows (e.g., PA fellows, nurse practitioner fellows), interns (e.g., psychology interns), auditioners, Advanced Practice Providers (APPs), and the like. While the disclosed embodiments are primarily described in reference to learners for a medical institution, other embodiments may provide automatic and optimized volume/scheduling for any personnel in any other field. The invention disclosed herein is not limited to the medical field.

As used herein, the term “preceptor” may refer to any teacher or instructor. For example, in a medical setting, the term “preceptor” may include an attending physician, an experienced advanced practice provider, a supervising psychologist, and the like. In other non-medical settings, the term “preceptor” may refer to any other person who is in a teaching or instructing role in their institution (e.g., senior accountant, senior engineer, partner attorney, and the like).

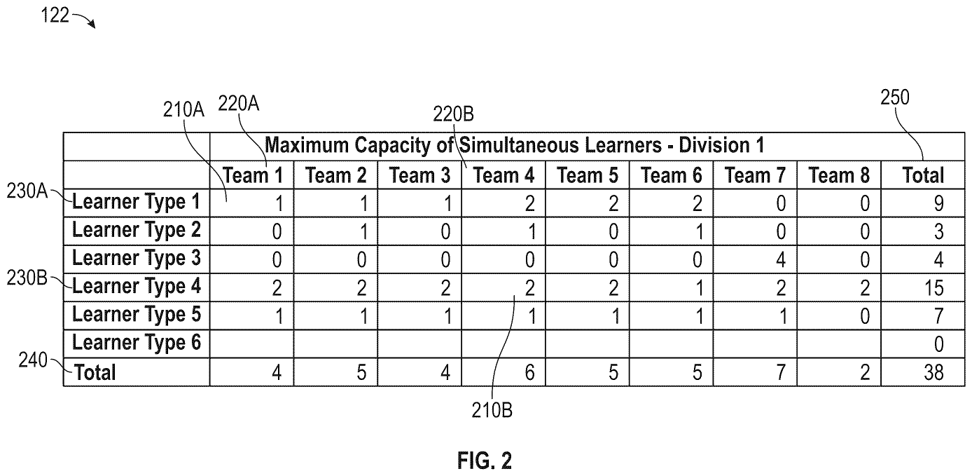

Maximum capacity of simultaneous learners 122 is input data for learner volume optimization module 120 that indicates a maximum number of learners that a group or a team (e.g., a team of a particular division) are able to accommodate. A particular example of maximum capacity of simultaneous learners 122 is illustrated in FIG. 2. In some embodiments, maximum capacity of simultaneous learners 122 is stored in memory 115 or any other appropriate database accessible to learner volume optimization module 120. In some embodiments, the data within maximum capacity of simultaneous learners 122 is provided by one or more users via a user interface on client system 130 or any other appropriate computer system. Each maximum capacity of simultaneous learners 122 as illustrated in FIG. 2 is associated with a particular group (e.g., team or division) of an institution (e.g., a mental health unit of a hospital campus, a cardiology center of the hospital campus, etc.).

As illustrated in FIG. 2, some embodiments of maximum capacity of simultaneous learners 122 include a learner capacity 210 (e.g., 210A, 210B . . . 210n) for each team 220 (e.g., 220A, 220B . . . 220n) and for each learner type 230 (e.g., 230A, 230B . . . 230n). For example, maximum capacity of simultaneous learners 122 of FIG. 2 includes a learner capacity 210A of “1” for team 220A (“Team 1”) and for learner type 230A (“Learner Type 1”). As another example, maximum capacity of simultaneous learners 122 includes a learner capacity 210B of “2” for team 220B (“Team 4”) and for learner type 230B (“Learner Type 4”). In some embodiments, maximum capacity of simultaneous learners 122 includes a total maximum capacity of simultaneous learners 240 for each team 220. In some embodiments, maximum capacity of simultaneous learners 122 includes a total maximum capacity of simultaneous learners 250 for each learner type 230. Each team 220 may be associated with a particular preceptor and may be assigned to a particular facility or area of the institution. Learner type 230 indicates the type or category of learner (e.g., students, residents, fellows, etc.). In some embodiments, learner type 230 may indicate a sub-type such as a first-year student, a second-year student, a third-year student, and the like. In some embodiments, the data within maximum capacity of simultaneous learners 122 may be provided by users via client systems 130.

In some embodiments, learner capacity 210 may be automatically weighted by learner volume optimization system 100 based on various factors such as available physical assets (e.g., workspace and computer workstations). For example, if physical assets data 126 (described below) indicates that a particular team has a large amount of physical space relative to other teams, learner volume optimization system 100 may automatically adjust learner capacity 210 to have a higher weighted value than other teams with less physical space. As another example, if physical assets data 126 indicates that a particular team has fewer number of computer workstations relative to other teams, learner volume optimization system 100 may automatically adjust learner capacity 210 to have a lower weighted value than other teams with more available computer workstations. Learner volume optimization system 100 may utilize any appropriate algorithm or method to automatically weight learner capacity 210 values within maximum capacity of simultaneous learners 122.

In some embodiments, learner volume optimization system 100 utilizes a Preceptor Independence and Oversight Goal (PIOG) value for learner capacity 210. In general, a PIOG value encompasses the amount of burden each preceptor would be subject to when overseeing learners. In some embodiments, each PIOG value is a function of: 1) the clinical independence of each learner; and 2) the amount of oversight each learner requires. In some embodiments, PIOG values may be calculated by adding the values assigned to each learner type in the independence and oversight columns shown below:

| Independence | | Oversight | |
|---|---|---|---|
| 0 | 3rd year medical students, | 0 | PGY5+ (fellows) |
| | APP students, psychology | | |
| | students | | |
| 1 | 4th yr medical students | 1 | PGY4 residents, post-doc |
| | | | psychologists |
| 2 | Post graduate year (PGY) | 2 | PGY3 residents |
| | 1 residents, psychology | | |
| | interns | | |
| 3 | PGY2 residents, APP | 3 | PGY2 residents |
| | fellows | | |
| 4 | PGY 3 residents | 4 | 4th yr medical students |
| 5 | PGY 4 residents, post-doc | 5 | 3rd yr medical students, |
| | psychologists | | psychology interns |
| 6 | PGY 5+ (fellows) | 6 | PGY1 residents, APP fellows, |
| | | | psychology students |

In some embodiments, the oversight side of the PIOG equation is different than the independence side because, for example, PGY1 residents and APP fellows can write orders/prescriptions, which creates more oversight burden for the preceptor than students (because students cannot write orders/prescriptions). Also, psychology students write reports that require more oversight than psychology interns or post-docs. In some embodiments, the ideal PIOG value for each preceptor (i.e., the optimal range by which the preceptor will have the optimal level of learners assigned to their team) ranges between 5-10, between 10-15, or any other appropriate range.

Anticipated learner data 124 is input data for learner volume optimization module 120 that indicates various data for learners that are to be scheduled with a preceptor/team. A particular example of anticipated learner data 124 is illustrated in FIGS. 3A and 3B. In some embodiments, anticipated learner data 124 is stored in memory 115 or any other appropriate database accessible to learner volume optimization module 120. In some embodiments, the data within anticipated learner data 124 is provided by one or more users via a user interface on client system 130 or any other appropriate computer system.

As illustrated in FIGS. 3A and 3B, some embodiments of anticipated learner data 124 include multiple entries 310 that are each associated with a particular learner that is to be scheduled (e.g., for a clinical experience) with a preceptor/team for a particular time period. In some embodiments, each entry 310 includes one or more of:
a month 320: indicates the month for which the learner needs to be scheduled
a date range 325: indicates the date range for which the learner needs to be scheduled
a rotation 330: indicates a rotation number for the learner (e.g., 01, 02, 03, etc.)
a half 335: indicates which half of the specified rotation time (e.g., 1^{St})
a learner ID 340: indicates a unique learner identifier for the learner
a day 345: indicates the day of the week for which the learner needs to be scheduled
a division 350: indicates the division to which the learner is to be assigned
a team 355: indicates which team to which the learner is to be assigned
a shift 360: indicates a time of the shift for which the learner needs to be scheduled
a location 365: indicates the specified physical location of the rotation (e.g., a building ID, etc.)
a learner type 370: indicates a type of learner (e.g., student, resident, fellow, etc.)
a learner name 375: indicates the name of the learner (may be filled in at a later date if unknown)
an academic year 380: indicates an ID of the academic year for the learner
a home institution 385: indicates a home institution (e.g., school, etc.) for the learner
a unique learner value 390: indicates an assigned value for the learner for the entry 310 that is based on amount of time the learner will be scheduled (e.g., a value of “0.5” if date 325 is less than a full month, a value of “1” if date 325 is a full month, etc.)

In some embodiments, each unique learner value 390 in anticipated learner data 124 may be automatically weighted or scaled by learner volume optimization system 100 based on the learner type 370. For example, students may have a higher weighted unique learner value 390 because they typically require more time of the preceptor. On the contrary, fellows may have a lower weighted unique learner value 390 because they typically require less time of the preceptor. Learner volume optimization system 100 may utilize any appropriate algorithm or method to automatically weight or scale unique learner values 390.

Physical assets data 126 is input data for learner volume optimization module 120 that includes various data for physical assets that are available to various preceptors/teams. In some embodiments, physical assets data 126 is stored in memory 115 or any other appropriate database accessible to learner volume optimization module 120. In some embodiments, the data within physical assets data 126 is provided by one or more users via a user interface on client system 130 or any other appropriate computer system.

In some embodiments, physical assets data 126 includes data about the physical space that is available to preceptors/teams. For example, the data about the physical space may indicate an amount of floor space (e.g., square footage), a number of rooms (e.g., meeting rooms, examination rooms, etc.), a number of computer workstations, or any other appropriate data that may indicate how much physical capacity or space is available for accommodating learners. In some embodiments, physical assets data 126 includes data about resources that are available to preceptors/teams. For example, the resource data may indicate the number of computer workstations available for accommodating learners.

Projected learner capacity 150 is generated by learner volume optimization module 120 and indicates, for each of a number of time periods (e.g., months), an available learner capacity for each team or group (e.g., within a division). In general, projected learner capacity 150 provides a quick overview of the learner capacity for each team in a division over certain time periods (e.g., months, weeks, etc.). In some embodiments, projected learner capacity 150 is stored in memory 115 or any other appropriate database/memory accessible to learner volume optimization module 120. A particular example of projected learner capacity 150 is illustrated in FIGS. 4A and 4B.

In some embodiments, projected learner capacity 150 is electronically transmitted via network 140 for display on client system 130. Each projected learner capacity 150 as illustrated in FIGS. 4A and 4B is associated with a particular division of an institution (e.g., a mental health unit of a hospital campus, a cardiology center of the hospital campus, etc.). In some embodiments, learner volume optimization module 120 may generate a projected learner capacity 150 for each division of the institution.

As illustrated in FIGS. 4A and 4B, some embodiments of projected learner capacity 150 may include a projected learner value 410 and an available learner capacity value 420 for each team 220 and for each time period 430. For example, for the month of July, Team 1 has a projected learner value 410 of “3.5” and an available learner capacity value 420 “0.5”. As another example, for the month of March, Team 3 has a projected learner value 410 of “5” and an available learner capacity value 420 “−1”. Each projected learner value 410 is a calculated amount of learners (e.g., from anticipated learner data 124 as described below) that need to be assigned to the particular team for the particular time period. Each available learner capacity value 420 indicates a calculated amount of learner capacity for the specific team for the specific time period. A positive number for available learner capacity value 420 may indicate that the specific team has capacity for learners for that specific time period. A negative or zero number for available learner capacity value 420 may indicate that the specific team does not have capacity for learners for that specific time period. Each time period 430 may be any appropriate time period such as a week, a month, and the like.

In some embodiments, available learner capacity value 420 within projected learner capacity 150 may have a visual indication (e.g., color, symbol, fill pattern, etc.) to indicate whether or not a team has available learner capacity for that particular time period 430. In the above example, for instance, the available learner capacity value 420 of “0.5” for Team 1 for the month of July may be a first color (e.g., green) to indicate that Team 1 has available learner capacity for that month. Similarly, the available learner capacity value 420 of “−1” for Team 3 for the month of March may be a second color (e.g., red) to indicate that Team 3 does not have available learner capacity for that month (i.e., the team is over-booked).

In some embodiments, projected learner capacity 150 includes a total projected learner value 411 that is a sum of projected learner values 410 for the particular time period 430. In some embodiments, projected learner capacity 150 includes a total available learner capacity value 421 that is a sum of available learner capacity values 420 for the particular time period 430. In some embodiments, total available learner capacity value 421 within projected learner capacity 150 may have a visual indication (e.g., color, symbol, fill pattern, etc.) to indicate whether or not the division (e.g., a sum of all teams 220) has available learner capacity for that particular time period 430.

In some embodiments, learner volume optimization system 100 may only display specific time periods 430 of projected learner capacity 150 based on user input. For example, instead of displaying all time periods 430 as illustrated in FIGS. 4A and 4B, some embodiments may only display projected learner values 410 and available learner capacity values 420 for the month of July if a user selects “July” as an input. This disclosure anticipates any appropriate display of information contained within projected learner capacity 150.

To calculate projected learner values 410 within projected learner capacity 150, some embodiments of learner volume optimization module 120 access and analyze anticipated learner data 124. More specifically, some embodiments locate and sum the relevant unique learner values 390 listed in anticipated learner data 124. For example, to calculate the projected learner values 410 of “3.5” for Team 1 for the month of July, learner volume optimization module 120 may sum all unique learner values 390 for entries 310 in anticipated learner data 124 for Team 1 (e.g., team 355=Team 1) and for July (e.g., month 320=July).

To calculate available learner capacity values 420 within projected learner capacity 150, some embodiments of learner volume optimization module 120 subtract the calculated projected learner value 410 for the specific team/time period from an input learner capacity for the specific team/time period. In some embodiments, the input learner capacity is the total maximum capacity of simultaneous learners 240 as discussed above in reference to FIG. 2. For example, to calculate the available learner capacity value 420 of “0.5” for Team 1 for the month of July, learner volume optimization module 120 may subtract the calculated projected learner value 410 of “3.5” from the total maximum capacity of simultaneous learners 240 of “4” for Team 1.

In some embodiments, a projected learner capacity dashboard 160 is generated and electronically displayed by learner volume optimization module 120. In general, projected learner capacity dashboard 160 indicates, for each of a number of time periods (e.g., months), an available learner capacity for each division within an institution. In general, projected learner capacity dashboard 160 is a quick and convenient way to view available learner capacity for multiple divisions within an institution. In some embodiments, projected learner capacity dashboard 160 is stored in memory 115 or any other appropriate database/memory accessible to learner volume optimization module 120. In some embodiments, projected learner capacity dashboard 160 is electronically transmitted via network 140 for display on client system 130. A particular example of projected learner capacity dashboard 160 is illustrated in FIGS. 5A and 5B.

As illustrated in FIGS. 5A and 5B, some embodiments of projected learner capacity dashboard 160 may include a projected learner value 510 and an available learner capacity value 520 for each division 350 and for each time period 430. For example, for the month of July, Division 1 has a projected learner value 510 of “5” and an available learner capacity value 520 “2”. As another example, for the month of April, Division 1 has a projected learner value 510 of “7.25” and an available learner capacity value 520 “−0.25”. Each projected learner value 510 is a total projected learner value 411 (as illustrated in FIGS. 4A and 4B) that is generated for the particular division. Each available learner capacity value 520 is a total available learner capacity value 421 (as illustrated in FIGS. 4A and 4B) that is generated for the particular division. A positive number for available learner capacity value 520 may indicate that the specific division has capacity for learners for that specific time period. A negative number for available learner capacity value 520 may indicate that the specific division does not have capacity for learners for that specific time period. Each time period 430 may be any appropriate time period such as a week, a month, and the like.

In some embodiments, available learner capacity value 520 within projected learner capacity dashboard 160 may have a visual indication (e.g., color, symbol, fill pattern, etc.) to indicate whether or not a division has available learner capacity for that particular time period 430. In the above example, for instance, the available learner capacity value 520 of “2” for Division 1 for the month of July may be a first color (e.g., green) to indicate that Division 1 has available learner capacity for that month. Similarly, the available learner capacity value 520 of “−0.25” for Division 1 for the month of April be a second color (e.g., red) to indicate that Division 1 does not have available learner capacity for that month.

In some embodiments, projected learner capacity dashboard 160 includes a total projected learner value 511 that is a sum of projected learner values 510 for the particular time period 430. In some embodiments, projected learner capacity dashboard 160 includes a total available learner capacity value 521 that is a sum of available learner capacity values 520 for the particular time period 430. In some embodiments, total available learner capacity value 521 within projected learner capacity dashboard 160 may have a visual indication (e.g., color, symbol, fill pattern, etc.) to indicate whether or not the institution (e.g., a sum of all divisions 350) has available learner capacity for that particular time period 430.

In some embodiments, learner volume optimization system 100 may only display specific time periods 430 of projected learner capacity dashboard 160 based on user input. For example, instead of displaying all time periods 430 as illustrated in FIGS. 5A and 5B, some embodiments may only display projected learner values 510 and available learner capacity values 520 for the month of July if a user selects “July” as an input. This disclosure anticipates any appropriate display of information contained within projected learner capacity dashboard 160.

In some embodiments, learner volume optimization system 100 provides a 3D learner capacity map 170 for display on client system 130. A particular example of 3D learner capacity map 170 is illustrated in FIG. 6A. In general, 3D learner capacity map 170 provides a three-dimensional view of an institution (e.g., a medical/hospital campus in this example) and the calculated learner capacity 610 for each preceptor/team for a selected time period (e.g., month). As illustrated in FIG. 6A, the learner capacities 610 are displayed proximate to the actual physical locations of their corresponding preceptors/teams. For example, if Team 1 from projected learner capacity 150 in FIGS. 4A and 4B is physically located on the top floor of the “General Impatient” division, the learner capacity 610 for Team 1 for the selected month (e.g., July 2025) is visually shown proximate to the top floor of the “General Impatient” division building on 3D learner capacity map 170. In some embodiments, 3D learner capacity map 170 is rotatable and tiltable to allow for users to better discern learner capacities 610.

In some embodiments, each learner capacity 610 in FIG. 6A corresponds to an available learner capacity value 420 of projected learner capacity 150. In some embodiments, each learner capacity 610 corresponds to a total available learner capacity value 421 of projected learner capacity 150. In some embodiments, learner capacities 610 within 3D learner capacity map 170 may have a visual indication (e.g., color, symbol, fill pattern, etc.) to indicate whether or not a team/division has available learner capacity for the selected time period. For example, learner capacity 610 may be:
a first color (e.g., green) if the corresponding available learner capacity value 420 is positive;
a second color (e.g., yellow) if the corresponding available learner capacity value 420 is zero; or
a third color (e.g., red) if the corresponding available learner capacity value 420 is negative.

In some embodiments, learner volume optimization module 120 may send one or more electronic alerts 180 (e.g., a text message, a push notification, and the like) to client system 130 (e.g., a smartphone, a computer, a tablet, etc.) to notify personnel of outputs of learner volume optimization module 120 (e.g., projected learner capacity 150, projected learner capacity dashboard 160, and 3D learner capacity map 170). For example, learner volume optimization module 120 may send an alert 180 to client system 130 that enables a user (e.g., via a hyperlink) to view all or part of projected learner capacity 150, projected learner capacity dashboard 160, and 3D learner capacity map 170. A user may view the alert and take any appropriate action or provide any requested input.

In some embodiments, learner volume optimization system 100 generates an alert 180 in response to a learner capacity being zero or negative. For example, if a particular available learner capacity value 420 is calculated to be negative (i.e., a preceptor/team has been over-scheduled for learners), an alert 180 may be automatically generated and sent to the administrator of learners, the preceptor, or any other person associated with the preceptor or team. The alert 180 may provide an indication of the available learner capacity value 420 and the particular associated time period. For example, the alert 180 may be “You have been over-scheduled for learners for the month of March by 1.5.” In some embodiments, the alert 180 may suggest re-balance recommendations.

In some embodiments, learner volume optimization system 100 may provide automatic evaluations for learners or physicians to evaluate a clinical experience of a learner. For example, an alert 180 may be automatically transmitted to a learner on or after the last day of the learner's clinical experience with a preceptor. The alert 180 may provide a link or other method to permit the learner to fill out an evaluation of their clinical experience. As another example, an alert 180 may be automatically transmitted to a preceptor on or after the last day of the learner's clinical experience. The alert 180 may provide a link or other method to permit the preceptor to fill out an evaluation of the learner.

In some embodiments, learner volume optimization system 100 may provide one or more alerts 180 to a learner about an upcoming clinical experience. As a first example, learner volume optimization system 100 may send an alert 180 to inform a learner about an upcoming scheduled clinical experience at a specific site. The alert 180 may include any appropriate information about the scheduled clinical experience (e.g., date, times, locations, etc.). As another example, learner volume optimization system 100 may send an alert 180 to a learner that provides map-based directions to a scheduled clinical site. As another example, learner volume optimization system 100 may send an alert 180 to a learner that provides goal/objectives for the learner for their specific scheduled clinical experience. As another example, learner volume optimization system 100 may send an alert 180 to a learner that provides orientation materials for their scheduled clinical experience. As another example, learner volume optimization system 100 may send an alert 180 to a learner that provides off-boarding procedures at the end of their clinical experience. As another example, learner volume optimization system 100 may send an alert 180 to a learner that provides the ability for the learner to request a letter of recommendation from their preceptor.

In some embodiments, learner volume optimization system 100 may provide one or more user interfaces that permit learners to interact with learner volume optimization system 100. For example, learner volume optimization system 100 may provide an interface that allows learners to request a clinical experience at a specific clinical site for a specific date/time. As another example, learner volume optimization system 100 may provide an interface that allows learners to signal that they would be applying to a particular residency/fellowship.

In some embodiments, learner volume optimization system 100 may provide the ability for preceptors or learner administrators to roll students forward. In some embodiments, learner volume optimization system 100 may provide the ability for preceptors or learner administrators to duplicate assignments from a previous year to the current year or a future year.

FIG. 6B is a chart illustrating a method 600 for optimizing learner volume at an institution. In some embodiments, method 600 may be utilized by the learner volume optimization system of FIG. 1. In some embodiments, method 600 may be performed by learner volume optimization module 120. At step 600A, method 600 accesses data indicating a maximum capacity of simultaneous learners for each team of a plurality of teams. In some embodiments, the indicating a maximum capacity of simultaneous learners is maximum capacity of simultaneous learners 122. In some embodiments, the team is team 220.

At step 600B, method 600 accesses anticipated learner data. In some embodiments, the anticipated learner data is anticipated learner data 124. In some embodiments, the anticipated learner data includes a plurality of learners to be scheduled and a unique learner value assigned to each particular learner of the plurality of learners to be scheduled. In some embodiments, the unique learner value is unique learner value 390.

In some embodiments, the unique learner value in step 600B is weighted or scaled based on a learner type (e.g., learner type 370). For example, a first learner type (e.g., students) may have a higher weighted unique learner value because they typically require more time of the preceptor. On the contrary, a second learner type (e.g., fellows) may have a lower weighted unique learner value because they typically require less time of the preceptor. Any appropriate scheme or rules may be utilized to weight or scale the unique learner value in step 600B. For example, step 600B may include accessing a plurality of weighting rules stored in memory that instruct how much each unique learner value should be weighted for each learner type.

In some embodiments, the unique learner value in step 600B that is assigned to each particular learner is based on an amount of time the particular learner needs to be scheduled. As an example, if a start date of a learner indicates that the learner will be assigned for a full month, the unique learner value in step 600B may be set to a first value (e.g., “1”). If, however, a start date of a learner indicates that the learner will be assigned for less than a full month, the unique learner value in step 600B may be set to a second value that is less than the first value (e.g., “0.5”).

At step 600C, method 600 automatically calculates a projected learner value for each particular team of a plurality of teams using the anticipated learner data. In some embodiments, the projected learner value of step 660C is projected learner value 410. In some embodiments, step 600C includes generating a projected learner capacity such as projected learner capacity 150. In some embodiments, the projected learner values generated in step 600C are for each division of an institution.

At step 600D, method 600 automatically calculates an available learner capacity for each particular team of the plurality of teams using the calculated projected learner value for each particular team. In some embodiments, the available learner capacity of step 660D is available learner capacity value 420.

At step 600E, method 600 displays, on an electronic display, the calculated available learner capacity for at least one team of the plurality of teams. In some embodiments, step 600E includes displaying a three-dimensional learner capacity map such as 3D learner capacity map 170. In some embodiments, step 600E includes displaying a projected learner capacity dashboard such as projected learner capacity dashboard 160. After step 600E, method 600 may end.

Particular embodiments may repeat one or more steps of the methods described herein, where appropriate. Although this disclosure describes and illustrates particular steps of particular methods as occurring in a particular order, this disclosure contemplates any suitable steps occurring in any suitable order. Moreover, although this disclosure describes and illustrates example methods that including particular steps, this disclosure contemplates any suitable method for performing the disclosed embodiments, which may include all, some, or none of the steps of particular methods descried herein, where appropriate. Furthermore, although this disclosure describes and illustrates particular components, devices, or systems carrying out particular steps of particular methods, this disclosure contemplates any suitable combination of any suitable components, devices, or systems carrying out any suitable steps of the disclosed methods.

FIG. 7 illustrates an example computer system 700 that can be utilized to implement aspects of the various methods and systems presented herein, according to particular embodiments. In particular embodiments, one or more computer systems 700 perform one or more steps of one or more methods described or illustrated herein. In particular embodiments, one or more computer systems 700 provide functionality described or illustrated herein. In particular embodiments, software running on one or more computer systems 700 performs one or more steps of one or more methods described or illustrated herein or provides functionality described or illustrated herein. Particular embodiments include one or more portions of one or more computer systems 700. Herein, reference to a computer system may encompass a computing device, and vice versa, where appropriate. Moreover, reference to a computer system may encompass one or more computer systems, where appropriate.

This disclosure contemplates any suitable number of computer systems 700. This disclosure contemplates computer system 700 taking any suitable physical form. As example and not by way of limitation, computer system 700 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, an augmented/virtual reality device, or a combination of two or more of these. Where appropriate, computer system 700 may include one or more computer systems 700; be unitary or distributed; span multiple locations; span multiple machines; span multiple data centers; or reside in a cloud, which may include one or more cloud components in one or more networks. Where appropriate, one or more computer systems 700 may perform without substantial spatial or temporal limitation one or more steps of one or more methods described or illustrated herein. As an example, and not by way of limitation, one or more computer systems 700 may perform in real time or in batch mode one or more steps of one or more methods described or illustrated herein. One or more computer systems 700 may perform at different times or at different locations one or more steps of one or more methods described or illustrated herein, where appropriate.

In particular embodiments, computer system 700 includes a processor 702, memory 704, storage 706, an input/output (I/O) interface 708, a communication interface 710, and a bus 712. Although this disclosure describes and illustrates a particular computer system having a particular number of particular components in a particular arrangement, this disclosure contemplates any suitable computer system having any suitable number of any suitable components in any suitable arrangement.

In particular embodiments, processor 702 includes hardware for executing instructions, such as those making up a computer program. As an example, and not by way of limitation, to execute instructions, processor 702 may retrieve (or fetch) the instructions from an internal register, an internal cache, memory 704, or storage 706; decode and execute them; and then write one or more results to an internal register, an internal cache, memory 704, or storage 706. In particular embodiments, processor 702 may include one or more internal caches for data, instructions, or addresses. This disclosure contemplates processor 702 including any suitable number of any suitable internal caches, where appropriate. As an example, and not by way of limitation, processor 702 may include one or more instruction caches, one or more data caches, and one or more translation lookaside buffers (TLBs). Instructions in the instruction caches may be copies of instructions in memory 704 or storage 706, and the instruction caches may speed up retrieval of those instructions by processor 702. Data in the data caches may be copies of data in memory 704 or storage 706 for instructions executing at processor 702 to operate on; the results of previous instructions executed at processor 702 for access by subsequent instructions executing at processor 702 or for writing to memory 704 or storage 706; or other suitable data. The data caches may speed up read or write operations by processor 702. The TLBs may speed up virtual-address translation for processor 702. In particular embodiments, processor 702 may include one or more internal registers for data, instructions, or addresses. This disclosure contemplates processor 702 including any suitable number of any suitable internal registers, where appropriate. Where appropriate, processor 702 may include one or more arithmetic logic units (ALUs); be a multi-core processor; or include one or more processors 702. Although this disclosure describes and illustrates a particular processor, this disclosure contemplates any suitable processor.

In particular embodiments, memory 704 includes main memory for storing instructions for processor 702 to execute or data for processor 702 to operate on. As an example, and not by way of limitation, computer system 700 may load instructions from storage 706 or another source (such as, for example, another computer system 700) to memory 704. Processor 702 may then load the instructions from memory 704 to an internal register or internal cache. To execute the instructions, processor 702 may retrieve the instructions from the internal register or internal cache and decode them. During or after execution of the instructions, processor 702 may write one or more results (which may be intermediate or final results) to the internal register or internal cache. Processor 702 may then write one or more of those results to memory 704. In particular embodiments, processor 702 executes only instructions in one or more internal registers or internal caches or in memory 704 (as opposed to storage 706 or elsewhere) and operates only on data in one or more internal registers or internal caches or in memory 704 (as opposed to storage 706 or elsewhere). One or more memory buses (which may each include an address bus and a data bus) may couple processor 702 to memory 704. Bus 712 may include one or more memory buses, as described below. In particular embodiments, one or more memory management units (MMUs) reside between processor 702 and memory 704 and facilitate accesses to memory 704 requested by processor 702. In particular embodiments, memory 704 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 704 may include one or more memories 704, where appropriate. Although this disclosure describes and illustrates particular memory, this disclosure contemplates any suitable memory.

In particular embodiments, storage 706 includes mass storage for data or instructions. As an example, and not by way of limitation, storage 706 may include a hard disk drive (HDD), a floppy disk drive, flash memory, an optical disc, a magneto-optical disc, magnetic tape, or a Universal Serial Bus (USB) drive or a combination of two or more of these. Storage 706 may include removable or non-removable (or fixed) media, where appropriate. Storage 706 may be internal or external to computer system 700, where appropriate. In particular embodiments, storage 706 is non-volatile, solid-state memory. In particular embodiments, storage 706 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 706 taking any suitable physical form. Storage 706 may include one or more storage control units facilitating communication between processor 702 and storage 706, where appropriate. Where appropriate, storage 706 may include one or more storages 706. Although this disclosure describes and illustrates particular storage, this disclosure contemplates any suitable storage.

In particular embodiments, I/O interface 708 includes hardware, software, or both, providing one or more interfaces for communication between computer system 700 and one or more I/O devices. Computer system 700 may include one or more of these I/O devices, where appropriate. One or more of these I/O devices may enable communication between a person and computer system 700. As an example, and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 708 for them. Where appropriate, I/O interface 708 may include one or more device or software drivers enabling processor 702 to drive one or more of these I/O devices. I/O interface 708 may include one or more I/O interfaces 708, where appropriate. Although this disclosure describes and illustrates a particular I/O interface, this disclosure contemplates any suitable I/O interface.

In particular embodiments, communication interface 710 includes hardware, software, or both providing one or more interfaces for communication (such as, for example, packet-based communication) between computer system 700 and one or more other computer systems 700 or one or more networks. As an example, and not by way of limitation, communication interface 710 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 710 for it. As an example, and not by way of limitation, computer system 700 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 700 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network, a Long-Term Evolution (LTE) network, or a 5G network), or other suitable wireless network or a combination of two or more of these. Computer system 700 may include any suitable communication interface 710 for any of these networks, where appropriate. Communication interface 710 may include one or more communication interfaces 710, where appropriate. Although this disclosure describes and illustrates a particular communication interface, this disclosure contemplates any suitable communication interface.

In particular embodiments, bus 712 includes hardware, software, or both coupling components of computer system 700 to each other. As an example and not by way of limitation, bus 712 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 712 may include one or more buses 712, where appropriate. Although this disclosure describes and illustrates a particular bus, this disclosure contemplates any suitable bus or interconnect.

Although the present disclosure and its advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

Moreover, the description in this patent document should not be read as implying that any particular element, step, or function can be an essential or critical element that must be included in the claim scope. Also, none of the claims can be intended to invoke 35 U.S.C. § 112(f) with respect to any of the appended claims or claim elements unless the exact words “means for” or “step for” are explicitly used in the particular claim, followed by a participle phrase identifying a function. Use of terms such as (but not limited to) “member,” “module,” “device,” “unit,” “component,” “element,” “mechanism,” “apparatus,” “machine,” “system,” “processor,” “processing device,” or “controller” within a claim can be understood and intended to refer to structures known to those skilled in the relevant art, as further modified or enhanced by the features of the claims themselves, and can be not intended to invoke 35 U.S.C. § 112(f). Even under the broadest reasonable interpretation, in light of this paragraph of this specification, the claims are not intended to invoke 35 U.S.C. § 112(f) absent the specific language described above.

While the include figures illustrate particular embodiments having particular components, this disclosure contemplates other embodiments having some or all of the described components, as well as additional components not described. Components of the present disclosure may be any suitable shape and may be in any suitable configuration.

As used in this document, “each” refers to each member of a set or each member of a subset of a set. Furthermore, as used in the document “or” is not necessarily exclusive and, unless expressly indicated otherwise, can be inclusive in certain embodiments and can be understood to mean “and/or.” Similarly, as used in this document “and” is not necessarily inclusive and, unless expressly indicated otherwise, can be inclusive in certain embodiments and can be understood to mean “and/or.”

The disclosure may be embodied in other specific forms without departing from the spirit or essential characteristics thereof. For example, each of the new structures described herein, may be modified to suit particular local variations or requirements while retaining their basic configurations or structural relationships with each other or while performing the same or similar functions described herein. The present embodiments are therefore to be considered in all respects as illustrative and not restrictive. Accordingly, the scope of the disclosures can be established by the appended claims rather than by the foregoing description. All changes which come within the meaning and range of equivalency of the claims are therefore intended to be embraced therein. Further, the individual elements of the claims are not well-understood, routine, or conventional. Instead, the claims are directed to the unconventional inventive concept described in the specification.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, functions, operations, or steps, any of these embodiments may include any combination or permutation of any of the components, elements, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative.

1. A system comprising:
one or more memory units configured to store:
data indicating a maximum capacity of simultaneous learners for each team of a plurality of teams; and
anticipated learner data comprising:
a plurality of learners to be scheduled; and
a unique learner value assigned to each particular learner of the plurality of learners to be scheduled;
one or more computer processors communicatively coupled to the one or more memory units, the one or more computer processors configured to perform program steps comprising:
accessing the data indicating the maximum capacity of simultaneous learners for each team of the plurality of teams;
accessing the anticipated learner data;
automatically calculating a projected learner value for each particular team of the plurality of teams using the anticipated learner data;
automatically calculating an available learner capacity for each particular team of the plurality of teams using the calculated projected learner value for each particular team; and
displaying, on an electronic display, the calculated available learner capacity for at least one team of the plurality of teams.

2. The system of claim 1, wherein the unique learner value assigned to each particular learner is based on an amount of time the particular learner needs to be scheduled.

3. The system of claim 1, wherein the program steps further comprise weighting or scaling each unique learner value assigned to each particular learner of the plurality of learners based on a learner type.

4. The system of claim 1, wherein the program steps further comprise electronically communicating a notification across an electronic communications network to one or more members of each particular team of a plurality of teams, the notification comprising:
the calculated available learner capacity for the particular team;
a hyperlink to view a three-dimensional learner capacity map; or
a hyperlink to view a projected learner capacity dashboard.

5. The system of claim 1, wherein displaying the calculated available learner capacity for at least one team of the plurality of teams comprises displaying a three-dimensional learner capacity map.

6. The system of claim 1, wherein displaying the calculated available learner capacity for at least one team of the plurality of teams comprises displaying a projected learner capacity dashboard.

7. The system of claim 1, wherein the one or more computer processors are further configured to execute machine-readable instructions, wherein the configuration of the one or more computer processors to execute the machine-readable instructions includes configuration to spawn more than one computer processes concurrently, wherein the concurrent more than one computer processes are configured to execute the machine-readable instructions to concurrently process data to perform the program steps.

8. A method by a computing system for optimizing learner volume at an institution, the method comprising:
accessing data indicating a maximum capacity of simultaneous learners for each team of a plurality of teams;
accessing anticipated learner data comprising:
a plurality of learners to be scheduled; and
a unique learner value assigned to each particular learner of the plurality of learners to be scheduled;
automatically calculating a projected learner value for each particular team of the plurality of teams using the anticipated learner data;
automatically calculating an available learner capacity for each particular team of the plurality of teams using the calculated projected learner value for each particular team; and
displaying, on an electronic display, the calculated available learner capacity for at least one team of the plurality of teams.

9. The method of claim 8, wherein the unique learner value assigned to each particular learner is based on an amount of time the particular learner needs to be scheduled.

10. The method of claim 8, further comprising weighting or scaling each unique learner value assigned to each particular learner of the plurality of learners based on a learner type.

11. The method of claim 8, further comprising electronically communicating a notification across an electronic communications network to one or more members of each particular team of a plurality of teams, the notification comprising:
the calculated available learner capacity for the particular team;
a hyperlink to view a three-dimensional learner capacity map; or
a hyperlink to view a projected learner capacity dashboard.

12. The method of claim 8, wherein displaying the calculated available learner capacity for at least one team of the plurality of teams comprises displaying a three-dimensional learner capacity map.

13. The method of claim 8, wherein displaying the calculated available learner capacity for at least one team of the plurality of teams comprises displaying a projected learner capacity dashboard.

14. The method of claim 8, further comprising spawning more than one computer processes concurrently, wherein the concurrent more than one computer processes are configured to execute machine-readable instructions to concurrently process data to perform the steps of the method.

15. One or more computer-readable non-transitory storage media embodying instructions that, when executed by a processor, cause the processor to perform operations comprising:
accessing data indicating a maximum capacity of simultaneous learners for each team of a plurality of teams;
accessing anticipated learner data comprising:
a plurality of learners to be scheduled; and
a unique learner value assigned to each particular learner of the plurality of learners to be scheduled;
automatically calculating a projected learner value for each particular team of the plurality of teams using the anticipated learner data;
automatically calculating an available learner capacity for each particular team of the plurality of teams using the calculated projected learner value for each particular team; and
displaying, on an electronic display, the calculated available learner capacity for at least one team of the plurality of teams.

16. The one or more computer-readable non-transitory storage media of claim 15, wherein the unique learner value assigned to each particular learner is based on an amount of time the particular learner needs to be scheduled.

17. The one or more computer-readable non-transitory storage media of claim 15, the operations further comprising weighting or scaling each unique learner value assigned to each particular learner of the plurality of learners based on a learner type.

18. The one or more computer-readable non-transitory storage media of claim 15, the operations further comprising electronically communicating a notification across an electronic communications network to one or more members of each particular team of a plurality of teams, the notification comprising:
the calculated available learner capacity for the particular team;
a hyperlink to view a three-dimensional learner capacity map; or
a hyperlink to view a projected learner capacity dashboard.

19. The one or more computer-readable non-transitory storage media of claim 15, wherein displaying the calculated available learner capacity for at least one team of the plurality of teams comprises displaying a three-dimensional learner capacity map.

20. The one or more computer-readable non-transitory storage media of claim 15, wherein displaying the calculated available learner capacity for at least one team of the plurality of teams comprises displaying a projected learner capacity dashboard.
